(19) United States
(12) Patent Application Publication
(10) Pub. No.: US 2022/0193255 A1
(43) Pub. Date: Jun. 23, 2022

(54) CHLORHEXIDINE SYSTEMS AND METHODS FOR OBTAINING SAME

(71) Applicant: Svitlana MOSKOVCHENKO, St-Laurent (CA)

(72) Inventor: Svitlana MOSKOVCHENKO, St-Laurent (CA)

(21) Appl. No.: 17/602,327

(22) PCT Filed: Apr. 8, 2020
(86) PCT No.: PCT/CA2020/050459

§ 371 Date: Oct. 8, 2021

Related U.S. Application Data
(60) Provisional application No. 62/830,684, filed on Apr. 8, 2019.

(51) Int. Cl. A61K 47/69; A01N 59/16; A61K 41/17; A01N 47/44; A61K 31/155

(52) U.S. Cl. CPC A61K 47/6929; A01N 59/16; B82Y 5/00; A01N 47/44; A61K 31/155; A61K 41/17

(57) ABSTRACT

The present technology generally relates to a chlorhexidine system comprising chlorhexidine or a salt thereof and metallic particles (such as silver and/or gold) wherein the chlorhexidine is conjugated to the surface of the metallic particles. Also described are methods for obtaining the system such as by gamma irradiation as well as the use of the system as an antimicrobial agent. Compositions comprising the chlorhexidine system and an additional component such as an alcohol or benzalkonium chloride and the use of these compositions as antimicrobials are also described.

### FIELD OF TECHNOLOGY

The present technology generally relates to chlorhexidine systems, to methods for obtaining such chlorhexidine systems as well as to uses thereof as antimicrobial agent.

### BACKGROUND INFORMATION

Chlorhexidine (CHD) and its salts are widely used as antiseptic and disinfectant in aqueous solutions. It is employed for skin disinfection, in wound dressings, in dentistry, for disinfection of surgical instruments and has applications in ophthalmology. The sterilization of chlorhexidine solutions cannot be accomplished by such a common and non-expensive way as gamma irradiation, because interaction with gamma rays leads to the degradation of chlorhexidine. The irradiation of aqueous solutions is associated with the emission of hydrated electrons and free OH and H radicals, which interact with chlorhexidine molecules and destroy them. Thus, more expensive and inconvenient autoclave sterilization techniques must be used by manufacturers during which chlorhexidine may still lose its strength causing a reduction of its antimicrobial efficiency.

Although chlorhexidine has shown good antimicrobial properties against the most bacteria tested in their free form, it is less effective against biofilms of several common bacteria (e.g. E. coli).

In view of the above, there is thus a need in the field for ways to protect chlorhexidine from degradation during its exposure to gamma irradiation while maintaining or improving its antimicrobial activity.

### SUMMARY OF TECHNOLOGY

In one aspect, the present technology relates to a chlorhexidine system comprising: metallic particles, the metallic particles having a core and a surface, and chlorhexidine or a salt thereof; wherein the chlorhexidine or the salt thereof is conjugated to the surface of the metallic particles.

In one aspect, the present technology relates to a composition comprising: the chlorhexidine system herein; and at least one additional component.

In one aspect, the present technology relates to a method for obtaining the chlorhexidine system as defined herein, the method comprising irradiating a mixture of metallic salts and the chlorhexidine or a salt thereof with gamma radiation.

In one aspect, the present technology relates to the use of the chlorhexidine system as defined herein as an antimicrobial.

In one aspect, the present technology relates to the use of the chlorhexidine system as defined herein for preventing or inhibiting growth of a biofilm.

In one aspect, the present technology relates to the use of the chlorhexidine system as defined herein for destruction of a biofilm.

In one aspect, the present technology relates to the use of the chlorhexidine system as defined herein as a disinfectant.

In one aspect, the present technology relates to the use of the chlorhexidine system as defined herein as an antiseptic.

In one aspect, the present technology relates to the use of the chlorhexidine system as defined herein as a skin disinfectant.

In one aspect, the present technology relates to the use of the chlorhexidine system as defined herein as a surface and equipment disinfectant.

In one aspect, the present technology relates to the use of the chlorhexidine system as defined herein for disinfection of surgical instruments.

### BRIEF DESCRIPTION OF THE DRAWINGS

FIG. 1 shows a graph of a UV-vis spectra of irradiated vs non-irradiated solution initially containing CHD=0.05 wt % and silver nitrate as precursor.

FIG. 2 shows a TEM image of silver nanoparticles formed by irradiation in presence of chlorhexidine gluconate from an aqueous solution of silver nitrate: presence of a conjugated layer on their surface is shown with arrows; the image made with TEN JEOL JEM 2100F.

FIG. 3 shows a TEM image of silver nanoparticles formed by irradiation in presence of chlorhexidine gluconate from an aqueous solution of silver nitrate: presence of a conjugated layer on their surface is clearly visible; the image made with FEI Tecnai G² F20 200 kV Cryo-STEM.

FIGS. 4A and 4B show TEM images of silver nanoparticles formed by irradiation in presence of chlorhexidine gluconate and polyvinyl alcohol from an aqueous solution of silver nitrate: FIG. 4A: irradiated at 7 kGy; FIG. 4B: irradiated at 3 kGy.

FIG. 5 shows a graph of a UV-vis spectra of irradiated solutions initially containing CHD=0.05 wt % and silver nitrate as precursor.

FIG. 6 shows UV-vis spectra of irradiated solutions initially containing different concentrations of chlorhexidine gluconate.

FIG. 7 shows UV-vis spectra of irradiated solutions initially containing CHD=0.075 wt % and different amounts of silver nitrate as precursor.

FIG. 8 shows normalized UV-vis spectra of irradiated solutions initially containing CHD=0.075 wt % and different amounts of silver nitrate as precursor.

FIGS. 9A, 9B and 9C show IBM images of silver nanoparticles formed by irradiation in presence of chlorhexidine gluconate and polyvinyl alcohol from an aqueous solution of silver nitrate irradiated at 7 kGy: FIG. 9A: at concentration of silver 60 ppm; FIG. 9B: 30 ppm; FIG. 9C: 15 ppm.

FIG. 10 is a photograph of Live/Dead E. coli ATCC25922 biofilm evaluation by confocal scanning laser microscopy after 10 min of exposure to the solution containing silver nanoparticles (Ag30 ppm-chlorhexidine gluconate 0.05 wt %-isopropanol 4 wt %) showing most of the biofilm dead (corresponding to red color).

FIG. 11 is a photograph of Live/Dead E. coli ATCC25922 biofilm evaluation by confocal scanning laser microscopy after 10 min of exposure to the solution not containing silver nanoparticles (chlorhexidine gluconate 0.05 wt %—isopropanol 4 wt %) showing most of the biofilm live (corresponding to green color).

FIG. 12 is graphs showing E. coli ATCC25922 biofilm mortality evaluation by confocal scanning laser microscopy after exposure to the solution not containing silver nanoparticles (chlorhexidine gluconate 0.05 wt %—isopropanol 4 wt %) versus exposure to the solution containing silver nanoparticles formed by gamma irradiation (Ag30 ppm-chlorhexidine gluconate 0.05 wt %—isopropanol 4 wt %).

FIG. 13 is a graph of a UV-vis spectra of irradiated vs non-irradiated solutions initially containing CHD=0.05 wt % and same amount of chloroauric acid as precursor.

FIG. 14 shows a TEM image of gold nanoparticles formed by irradiation in presence of chlorhexidine gluconate from an aqueous solution of chloroauric acid: quasi-spherical and star-shaped nanoparticles; the image made with FEI Tecnai G² F20 200 kV Cryo-STEM.

FIG. 15 shows a TEM image of gold nanoparticles formed by irradiation in presence of chlorhexidine gluconate from an aqueous solution of chloroauric acid: presence of a conjugated layer on their surface; the image made with FEI Tecnai G² F20 200 kV Cryo-STEM.

### DETAILED DESCRIPTION OF TECHNOLOGY

Before continuing to describe the present disclosure in further detail, it is to be understood that this disclosure is not limited to specific compositions or process steps, as such may vary. It must be noted that, as used in this specification and the appended embodiments, the singular form “a”, “an” and “the” include plural referents unless the context clearly dictates otherwise.

As used herein, the term “about” in the context of a given value or range refers to a value or range that is within 20%, within 10%, and more within 5% of the given value or range.

It is convenient to point out here that “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

In one embodiment, the present technology provides a chlorhexidine system wherein the chlorhexidine is protected from degradation during sterilization. The chlorhexidine of the present technology also possesses antimicrobial activity rendering it efficient for preventing growth and/or proliferation of biofilms.

In one embodiment, the chlorhexidine system of the present technology comprises particles made of metals, preferably transition metals (e.g., metallic elements occupying a central block (Group IVB-VIII, IB, and IIB, or 4-12) in the periodic table). The metallic particles have a surface which is in contact with the exterior environment and have a core. In some instances, the metallic particles of the present technology are formed from metallic salts. In some further instances, the metallic particles of the present technology are formed from metallic salts by irradiation, preferably gamma irradiation.

The chlorhexidine system further comprises chlorhexidine or a salt thereof (e.g., chlorhexidine di-gluconate, acetate and chloride). In some instances, the chlorhexidine or a salt thereof is conjugated to the surface of the metallic particles. As used herein, the term “conjugated” refers to a system that has a region of their orbitals (e.g., p-orbitals) that overlap. In some instances, the metallic particles are metallic nanoparticles having an average size ranging from between about 1 nm and about 1000 nm, or between about 1 nm and about 750 nm, or between about 1 nm and about 500 nm, or between about 1 nm and about 250 nm, or between about 1 nm and about 100 nm. As used herein, the term “size” refers to the largest dimension of the particles.

Particles as defined herein are not limited to any particular geometric shape and can for example be in the form of globules, bits, droplets, may have a spherical shape, an elliptical shape or may have an irregular or discontinuous shape. The shape of the particles may be irregular so as to create physical attachment points or locations to assist with retention of the particles into or onto a substrate. The surface of the particles or parts thereof may be irregular, discontinuous and/or rough. Particles such as nanoparticles, may be visualized using techniques such as, but not limited to, extraction method with tracer techniques (e.g., electron microscopy). Other techniques to visualize particles will be known to those of skill in the art. The size of the particle is determined by techniques well known in the art, such as, but not limited to, photon correlation spectroscopy, laser diffractometry, scanning electron microscopy and/or 3CCD (charged-couple device).

In some embodiments, the metallic particles are made of silver (Ag) and/or oxides thereof. In some instances, the silver particles of the present technology are silver nanoparticles. In some instances, the particles of the present technology are prepared from silver (Ag) and/or oxides thereof using irradiation. In some other instances, the silver nanoparticles of the present technology may be prepared according to various methods. One method for silver nanoparticle synthesis uses nucleation of particles within a solution. This nucleation occurs when a silver ion complex, usually AgNO₃ or AgClO₄, is reduced to colloidal silver in the presence of a reducing agent. When the concentration increases enough, dissolved metallic silver ions bind together to form a stable surface. The surface is energetically unfavorable when the cluster is small, because the energy gained by decreasing the concentration of dissolved particles is not as high as the energy lost from creating a new surface. When the cluster reaches a certain size, known as the critical radius, it becomes energetically favorable, and thus stable enough to continue to grow. This nucleus then remains in the system and grows as more silver atoms diffuse through the solution and attach to the surface. When the dissolved concentration of atomic silver decreases enough, it is no longer possible for enough atoms to bind together to form a stable nucleus. At this nucleation threshold, new nanoparticles stop being formed, and the remaining dissolved silver is absorbed by diffusion into the growing nanoparticles in the solution. As the particles grow, other molecules in the solution diffuse and attach to the surface. This process stabilizes the surface energy of the particle and blocks new silver ions from reaching the surface. The attachment of these capping/stabilizing agents slows and eventually stops the growth of the particle. The most common capping ligands are trisodium citrate and polyvinylpyrrolidone (PVP), but many others are also used in varying conditions to synthesize particles with particular sizes, shapes, and surface properties. Other methods of preparing silver nanoparticles include, but are not limited to, the use of reducing sugars, citrate reduction, reduction via sodium borohydride, the silver mirror reaction, the polyol process, seed-mediated growth, and light-mediated growth. Each of these methods, or a combination of methods, offer different degrees of control over the size distribution as well as distributions of geometric arrangements of the nanoparticle. Another method for synthesizing silver nanoparticles is citrate reduction. Citrate reduction involves the reduction of a silver source particle, usually AgNO₃ or AgClO₄, to colloidal silver using trisodium citrate, Na₃C₆H₅O₇. The synthesis is usually performed at an elevated temperature (˜100° C.) to maximize the monodispersity (uniformity in both size and shape) of the particle. In this method, the citrate ion traditionally acts as both the reducing agent and the capping ligand, making it a useful process for AgNP production due to its relative ease and short reaction time. The silver particles formed may exhibit broad size distributions and form several different particle geometries simultaneously. The addition of stronger reducing agents to the reaction is often used to synthesize particles of a more uniform size and shape.

In some embodiments, the stabilizing agent used in the preparation of silver nanoparticles is selected from: carboxymethylcellulose (CMC), polyethylene glycol (PEG), polyvinylpyrrolidone (PVP), polyvinyl alcohol (PVA), polyethyleneimine (PEI), propylene glycol (PG), dodecanoic acid (DDA), polyacrylic acid (PAA), chitosan, pectin, alginate, gelatin, starch, gums (such as karaya gum, gum arabic, or the like), cyclodextrins, cetyltrimethylammonium bromide (CTAB), sodium dodecyl sulfate (SDS), cationic and anionic ligands, and other polymers, proteins, oligosaccharides, phenolics and flavonoids, of synthetic and natural origin, including the organic extracts derived from plants, known to stabilize the size of metallic particles in the process of reduction from the metallic salts in such a way, that metallic particles remain in the size range of between about 1 nm and about 1000 nm.

In some embodiments, the reducing agent used in the preparation of silver nanoparticles is selected from: borohydrides (e.g., sodium borohydride), citrates (e.g., sodium citrate), tannic acid and ascorbic acids and the salts thereof, formates (e.g., ammonium formate), ethylene glycol, polyols, N,N-dimethylformamide (DMF), hydrazine hydrate, hydroquinone and the salts thereof were used as reducing agents.

Is some other embodiments, the metallic particles are made of gold (Au). In some instances, the gold particles of the present technology are gold nanoparticles. In some instances, the particles of the present technology are prepared from gold-containing salts using irradiation. In some other instances, the, gold nanoparticles are produced in a liquid by reduction of chloroauric acid (H[AuCl₄]). To prevent the particles from aggregating, stabilizing agents are added. Citrate acts both as the reducing agent and colloidal stabilizer. Other methods may be used to prepare gold nanoparticles such as, for example, the Turkevich method, by use of capping agents, the Brust-Schiffrin method, the Perrault method, the Martin method, the Navarro method, by sonolysis, the block-copolymer-mediated methods, which are all known in the art. In some other embodiments, the metallic particles are made of a mixture of silver and gold. In some instances, the particles made of a mixture of silver and gold may be made as an alloy with different weight % of silver-to-gold.

In some other instances, the particles made of a mixture of silver and gold may comprise a layered structure of gold layers or spheres and silver layers or spheres. In some of these instances, the silver layer or sphere may cover the gold layer or sphere, whereas in other instances it may be the gold layer or sphere that covers the silver layer or sphere. In other instances, the silver and gold layers or spheres may be disposed in alternation. The composition of such particles depends on the quantity and proportion of reducing-stabilizing agents and gold and silver precursors, as well as the order of reduction.

In one embodiment, the present technology relates to a method for obtaining the chlorhexidine system as defined herein. The method comprises forming a mixture of the metallic salts and the chlorhexidine of the salts thereof and irradiating the mixture. The irradiation step allows to conjugate the chlorhexidine or the salt thereof to the surface of the metallic particles. In some instances, the irradiation is performed with gamma radiation (gamma rays). The gamma rays are used in an amount ranging between about 1 kGy and about 50 kGy, which are dose levels commonly used for sterilization.

In some embodiments, the method of preparing the chlorhexidine system of the present technology provide a rate of preservation of chlorhexidine of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. As used herein, the expression “rate of preservation of chlorhexidine” refers to the % of chlorhexidine of salts thereof present in the mixture that is conjugated to the metallic particles upon irradiation of the mixture.

In some implementations, the conjugation of the chlorhexidine to the metallic core protects the chlorhexidine from degradation during its exposure to irradiation while retaining the chlorhexidine's antimicrobial activity.

In one embodiment, the chlorhexidine system of the present technology is used as an antimicrobial agent.

In one embodiment, the chlorhexidine system of the present technology is used as disinfectant.

In one embodiment, the chlorhexidine system of the present technology is used to inhibit growth and/or proliferation of biofilms.

In one embodiment, the chlorhexidine system of the present technology is used to cause mortality of biofilms.

In one embodiment, the present technology also relates to composition comprising the chlorhexidine system as defined herein. The compositions of the present technology may be used as a disinfectant, as antimicrobial and/or to inhibit growth and/or proliferation of biofilms.

In some instances, the composition is an aqueous composition and is prepared by dissolving the chlorhexidine system of the present technology in water. In an embodiment, a composition disclosed herein comprises an amount of the chlorhexidine system as defined herein that provides a desired beneficial effect to a composition disclosed herein. In aspects of this embodiment, a composition disclosed herein comprises the chlorhexidine system in an amount of, e.g., about 0.01%, about 0.02%, about 0.03%, about 0.04%, about 0.05%, about 0.06%, about 0.07%, or about 0.08%, about 0.09% by weight of the composition. In other aspects of this embodiment, a composition disclosed herein comprises chlorhexidine system in an amount of between about 0.01% and about 1.0% by weight of the composition. In other aspects of this embodiment, a composition disclosed herein comprises chlorhexidine system in an amount of between about 0.01% and about 2.0% by weight of the composition. In other aspects of this embodiment, a composition disclosed herein comprises chlorhexidine system in an amount of between about 0.01% and about 5.0% by weight of the composition. In other aspects of this embodiment, a composition disclosed herein comprises chlorhexidine system in an amount of between about 0.01% and about 10.0% by weight of the composition.

In one embodiment, the irradiated chlorhexidine system of the present technology may be stable for several months without precipitating. In one embodiment, the irradiated chlorhexidine system of the present technology may be stable for several years without precipitating. In one embodiment, the irradiated chlorhexidine system of the present technology may be stable for several months without degradation of chlorhexidine. In one embodiment, the irradiated chlorhexidine system of the present technology may be stable for several years without degradation of chlorhexidine. In one embodiment, the irradiated chlorhexidine system of the present technology may be stable for several months without precipitating and without degradation of chlorhexidine. In one embodiment, the irradiated chlorhexidine system of the present technology may be stable for several years without precipitating and without degradation of chlorhexidine.

The chlorhexidine system of the present technology may be used in disinfectants (disinfection of the skin and hands and surfaces), cosmetics (additive to creams, toothpaste, deodorants, and antiperspirants), and pharmaceutical products (preservative in eye drops, active substance in wound dressings and antiseptic mouthwashes). The chlorhexidine system of the present technology may also be used in endodontics, for example in for root canal irrigation and as an intracanal dressing.

The chlorhexidine system of the present technology is active against Gram-positive and Gram-negative organisms, facultative anaerobes, aerobes, and yeasts. Use of the chlorhexidine system of the present technology may be used in mouthwash in combination with normal tooth care can help reduce the build-up of plaque and improve mild gingivitis. The chlorhexidine system of the present technology may be used as a skin cleanser for surgical scrubs, a cleanser for skin wounds, for preoperative skin preparation and germicidal hand rinses. Chlorhexidine eye drops have been used as a treatment for eyes affected by Acanthamoeba keratitis.

The chlorhexidine system of the present technology may be used alone and may be mixed with additional components such as with suitable diluent, excipient or solvent to form compositions or formulations comprising the chlorhexidine system of the present technology. Examples of additional components include, but are limited to: alcohols (ethanol and isopropyl alcohol) and benzalkonium chloride which are typical used for disinfection of skin, of wounds, of surfaces, instruments and medical devices by application and letting to dry, or according to the application procedure and approved guidelines for each system.

### EXAMPLES

The examples below are given so as to illustrate the practice of various embodiments of the present disclosure. They are not intended to limit or define the entire scope of this disclosure. It should be appreciated that the disclosure is not limited to the particular embodiments described and illustrated herein but includes all modifications and variations falling within the scope of the disclosure as defined in the appended embodiments.

### Example 1—Preparation of CHD-Coated Silver Particles by Irradiation Method (Trial 1)

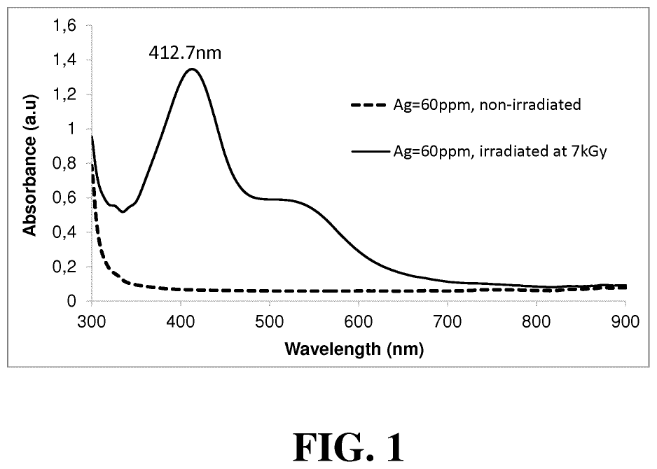

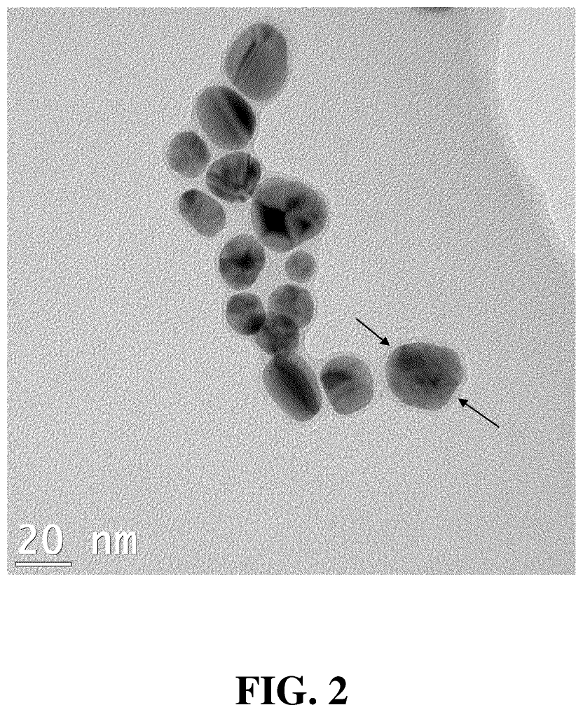

An aqueous solution of silver nitrate salt (as the source of silver) was prepared so that the final concentration of silver in the solution was 60 ppm. A 20 wt % aqueous solution of chlorhexidine gluconate (CHD) was added to make the resulting concentration of 0.05 wt %. Finally, isopropanol was added to achieve the concentration of 4 wt % in the resulting solution. The sample was a transparent colorless liquid. Thermo Scientific Evolution 220 Spectrophotometer was used to monitor the absorbance spectra of the sample, which is shown in FIG. 1 as the dashed line. The 30 ml sample solution was then subjected to irradiation by gamma rays at 7 kGy. The resulting solution was a transparent brownish liquid showing a clear peak of absorption at the wavelength 412.7 nm (solid line in FIG. 1), which corresponds to the presence of silver nanoparticles. The sample of the irradiated solution was presented for imaging to Transmission Electron Microscope (JEOL JEM 2100F) and an example of the image is shown in FIG. 2, which confirms formation of silver nanoparticles in the irradiated solution. The nanoparticles have quasi-spherical form and a visible conjugated layer around their surface.

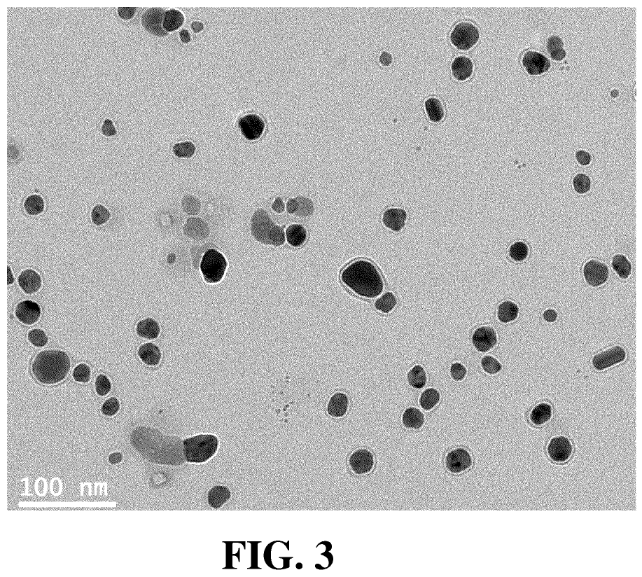

After the formation of silver nanoparticles has been confirmed, the concentration of chlorhexidine gluconate in the irradiated solution was measured using high performance liquid chromatography (HPLC) and it was determined as 0.0295 wt %, which demonstrates a rate of preservation of 59% compared to the initial level in the sample before irradiation. For comparison, the same sample was sent for imaging with FEI Tecnai G² F20 200 kV Cryo-STEM Transmission Electron Microscope, and the picture is presented in FIG. 3. The presence of the conjugated layer around the nanoparticles is visible even more clearly.

### Example 2—Preparation of CHD-Coated Silver Particles by Irradiation Method (Trial 2)

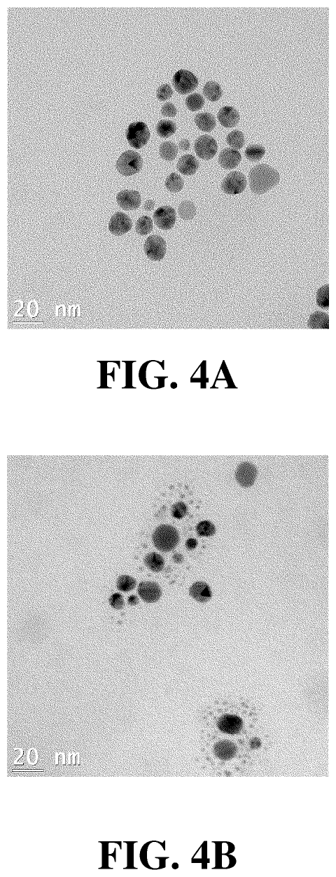

Two identical samples were prepared as described in Example 1, each of them being colorless transparent aqueous solutions containing 60 ppm of silver in the form of silver nitrate, 0.5 wt % of polyvinyl alcohol, 4 wt % of isopropanol and 0.05 wt % of chlorhexidine gluconate. The samples were subjected to different doses of gamma irradiation—the first sample to 7 kGy and the second to 3 kGy. After irradiation the color of the samples changed to the transparent brown. Chlorhexidine gluconate was measured using HPLC and was determined as 0.0294 wt % in the sample irradiated by 7 kGy and 0.039 5 wt % in the sample irradiated by 3 kGy, meaning a rate of preservation of 58.8% and 79% respectively. The TEM images of both samples are presented in FIGS. 4A and 4B, where the presence of significantly smaller nucleation centers (seeds) can be noticed in FIG. 4B, corresponding to the sample which received smaller irradiation dose of 3 kGy (FIG. 4A).

### Example 3—Influence of Stabilizing Agent on CHD-Coated Silver Particles

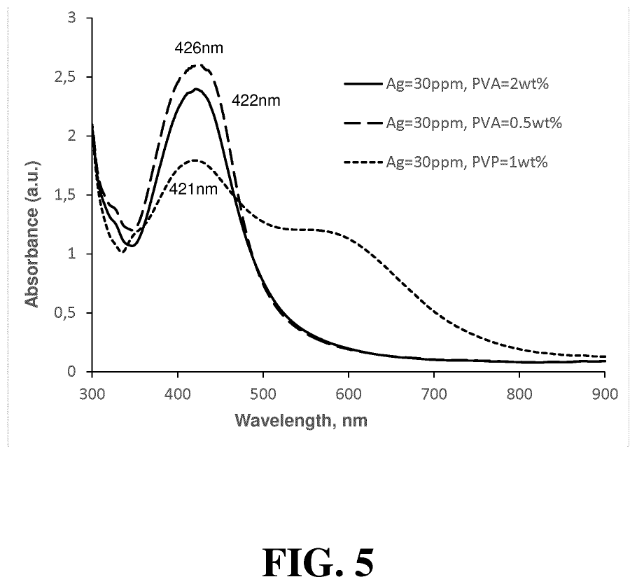

To evaluate any influence of the amount and the nature of the stabilizing agent, three different samples were prepared as described in Example 1. Each of them contained 30 ppm of silver in the form of silver nitrate and 0.05 wt % of chlorhexidine gluconate in an aqueous solution. The first sample additionally contained 0.5 wt % of polyvinyl alcohol (PVA) and 10 wt % of isopropanol, the second—2 wt % of polyvinyl alcohol and 10 wt % of isopropanol and the third sample additionally contained 1 wt % of polyvinylpyrrolidone (PVP) and 4 wt % of isopropanol. All three samples represented clear colorless liquids before irradiation. The irradiated at 10 kGy samples changed their color to the transparent brown color of different intensities. The concentration of chlorhexidine gluconate was measured using HPLC and determined as 0.015 wt % in the first and the second sample and 0.016 wt % in the third sample. The UV-vis spectra of all three samples show formation of silver nanoparticles, with the only difference that in the sample which contained PVP the nanoparticles are larger than in those which contained PVA (FIG. 5; the “shoulder” of the small dashed line indicates presence of nanoparticles larger than 100 nm).

### Example 4—Influence of CHD Concentration on CHD-Coated Silver Particles

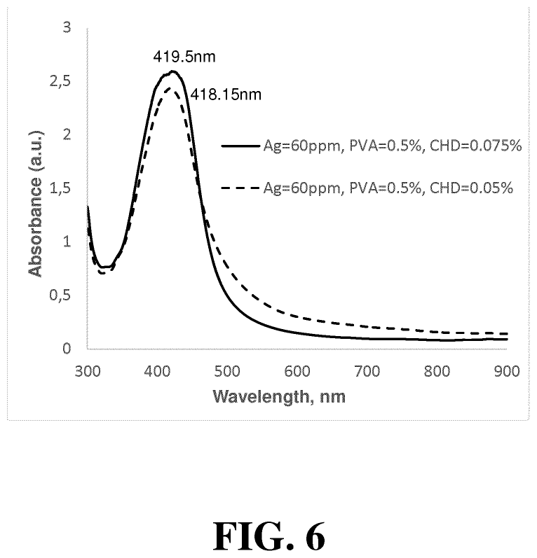

To evaluate any difference caused by the initial amount of chlorhexidine gluconate, two samples were prepared as in Example 1, but one of them contained 0.05 wt % of chlorhexidine gluconate and another sample contained 0.075 wt % of chlorhexidine gluconate before irradiation. Both samples were irradiated at 7 kGy, by the action of which the nanoparticles of silver were formed in both samples. The concentration of chlorhexidine gluconate after irradiation was measured using HPLC and was determined as 0.0294 wt % in the first sample and 0.052 wt % in the second one, showing the preservation rate of 58.8% and 69.3% respectively. The analysis of UV-vis scans of the samples (FIG. 6) shows that more nanoparticles were formed in the case when the solution contained more chlorhexidine gluconate (higher peak) and they were a bit larger (419.5 nm for the wavelength corresponding to the peak of absorbance for initial level of CHD=0.075% versus 418.15 nm for CHD=0.05%. Shifting the peak to the side of larger wavelengths normally indicates the presence of larger nanoparticles).

### Example 5—Influence of Silver Concentration on CHD-Coated Silver Particles

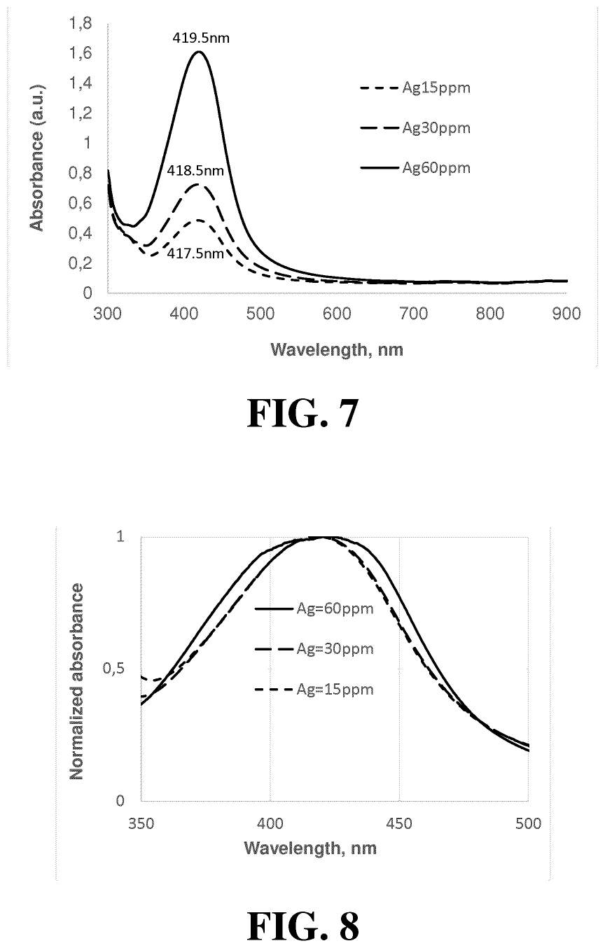

To evaluate the influence of the amount of silver present in the form of a silver salt as a precursor for nanoparticles formation, three different samples were prepared as described in Example 1. Each of them contained 0.075 wt % of chlorhexidine gluconate, 0.5 wt % of polyvinyl alcohol and 4 wt % of isopropanol in an aqueous solution. Silver nitrate was added to each of the samples so that the concentration of silver in the samples was 15 ppm, 30 ppm and 60 ppm. All the samples were transparent colorless solutions. They were subjected to gamma irradiation at 7 kGy and the concentration of chlorhexidine gluconate was measured consequently using HPLC. After irradiation, all the samples had the appearance of brownish transparent liquids, and the color was more intensive in the samples containing more silver. Formation of silver nanoparticles was confirmed by UV-vis analysis, showing the maximum of absorption at the wavelengths characteristic for the formation of silver nanoparticles (FIG. 7). The spectra corresponding to higher concentration of silver precursor are indicating formation of larger amounts of silver nanoparticles (having the higher peaks of absorbance) and the presence of slightly larger nanoparticles (the wavelengths corresponding to the peaks of absorbance are shifted to the side of larger wavelengths). Chlorhexidine gluconate was detected in all the irradiated samples, showing a rate of preservation from 57.33% to 69.33% with the concentration of silver increasing from 15 ppm to 60 ppm (Table 1).

TABLE 1
The characteristics of the irradiated solutions
related to the concentration of silver.

| Ag, ppm | 60 | 30 | 15 |
|---|---|---|---|
| Chlorhexidine gluconate after | 0.052 | 0.049 | 0.043 |
| irradiation, % | | | |
| Preservation, % | 69.33 | 65.33 | 57.33 |
| Max. absorbance (a.u.) of a | 1.608154 | 0.724078 | 0.484304 |
| double diluted sample | | | |
| Wavelength corresponding to | 419.5 | 418.5 | 417.5 |
| the max. of absorbance, nm | | | |

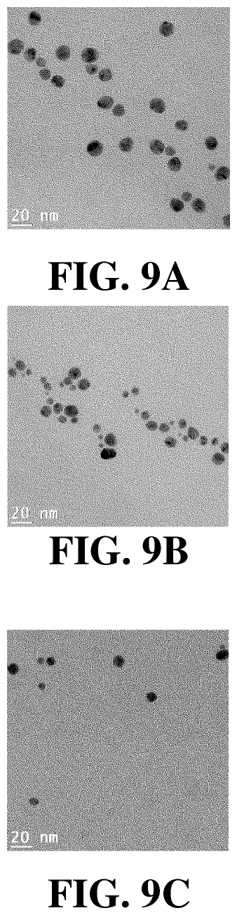

To evaluate the polydispersity of the irradiated samples, UV-vis scans of diluted samples were normalized as shown in FIG. 8, and the monodispersity was evaluated as a peak width corresponding to the half of the maximum of absorbance. The broadest spectrum corresponds to Ag=60 ppm, showing that with increasing concentration of silver salt in the samples, more polydisperse nanoparticles are formed during irradiation. Three TEM images corresponding to different concentration of silver are presented in FIGS. 9A, 9B and 9C. It can be noticed, that the nanoparticles have almost the same morphology independently of silver concentration, but at a lowest concentration of 15 ppm there are less nanoparticles present and they are slightly smaller, which is consistent with the conclusions based on the analysis of UV-vis spectra. Higher concentrations of silver precursor lead to the formation of larger nanoparticles, with all the other conditions being the same.

### Example 7—Assessment of CHD-Coated Silver Particles Antimicrobial Activity

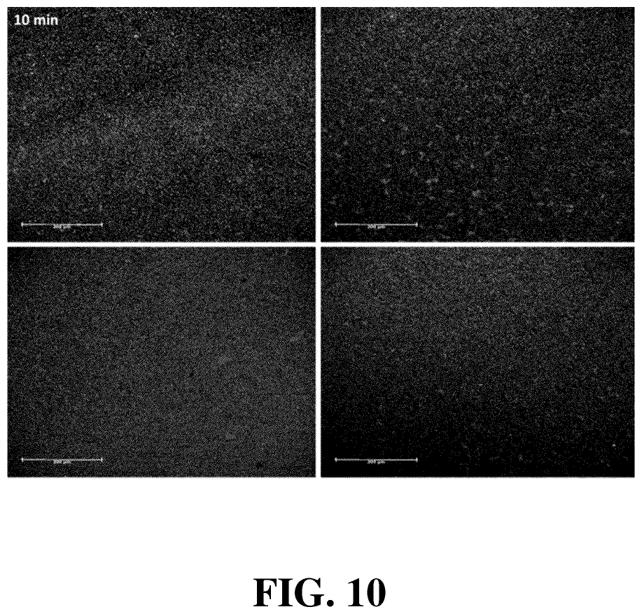

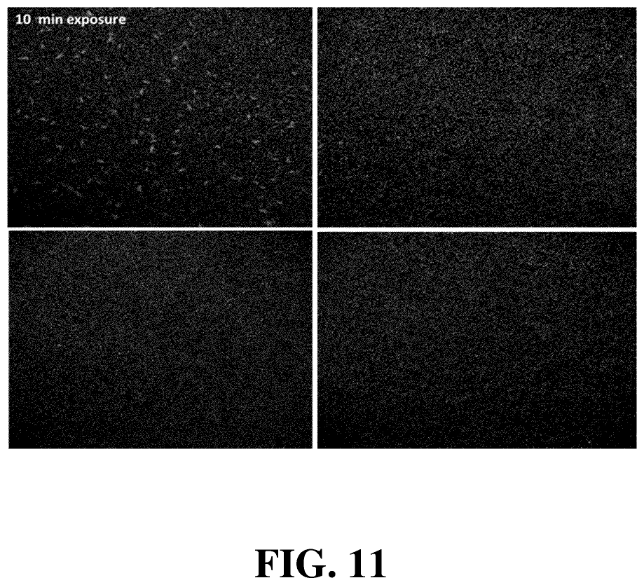

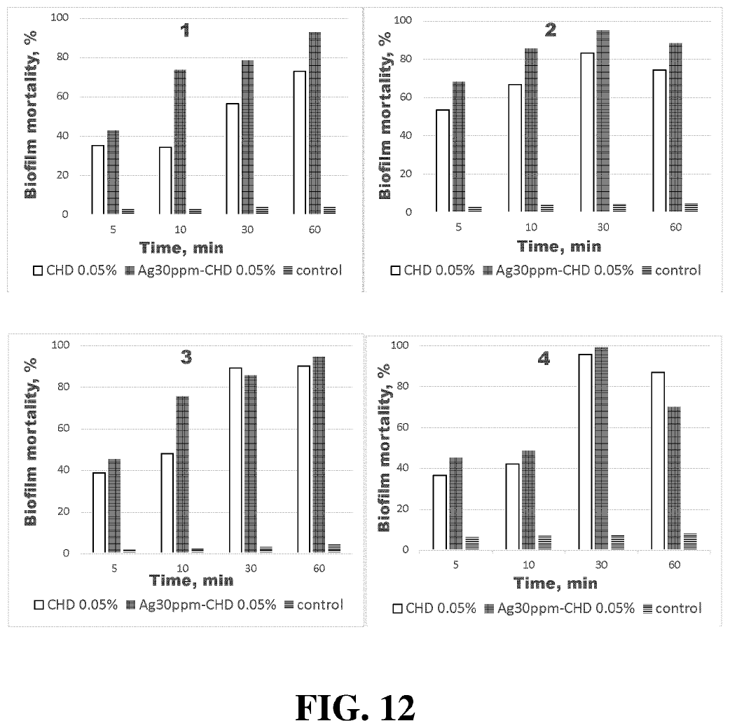

Bacterial strain Escherichia coli ATCC 25922 was used for biofilm mortality evaluation. The strain was cultured in Tryptic Soy Broth (TSB) and incubated at 37° C. overnight. An overnight culture of E. coli ATCC 25922 was then diluted 100-fold in TSB, and thereafter the cells were grown on the wells of 8-well chambered cover glasses during 24 h at 37° C., forming the biofilms. The culture supernatant was removed, and a fresh TSB medium containing 400 μl of the testing solution was added on top of the biofilms and the biofilms were further incubated during the time of exposure at 30° C. When the exposure period was over, the testing solution from the top of the biofilm from each cover glass was removed and analyzed by using Live/Dead BackLight Bacterial Viability and Counting Kit (Invitrogen, Molecular Probes) with a confocal laser microscope (Leica model TCS SPS; Leica Microsystems CMS GmbH, Mannheim, Germany) using a 20× dry objective (HC PL FLUOTAR 20.0×0.50 DRY). The images of Live/Dead biofilms after the exposure time of 10 min for a) solution prepared as described in Example 5 having the concentration of silver of 30 ppm (Ag30 ppm-chlorhexidine gluconate 0.05 wt %—isopropanol 4 wt %), b) the same solution which did not contain any silver (chlorhexidine gluconate 0.05 wt %—isopropanol 4 wt %) are presented in FIG. 10 and FIG. 11 respectively. Green color in the images means alive biofilms, and red color in the images means dead biofilms. The images taken at different spots of the biofilm were analyzed using ImageJ software, which allowed to calculate biofilm mortality. The experiment was repeated 4-times and the results for 4 replicas are presented in FIG. 12, showing the comparison of biofilm mortality caused by the conventional solution non-containing silver nanoparticles (chlorhexidine gluconate 0.05 wt %—isopropanol 4 wt %) and the same solution containing silver nanoparticles, formed by gamma irradiation.

### Example 8—Comparative—Irradiation of CHD-Coated Gold Particles Prepared by Chemical Method

Gold nanoparticles were synthesized using chloroauric acid (HAuCl₄*3H₂O, 1 wt % solution in water) as a precursor and ascorbic acid as the reducing agent so that the molar ratio gold/ascorbic acid was 1:10. Chloroauric acid was added into the aqueous solution of ascorbic acid kept at ambient temperature by continuous stirring at 700 rpm. Right after the addition the solution became violet and soon after its color changed to red. After 1 minute of mixing, solution of chlorhexidine gluconate (20 wt %) was added so that its final concentration in the solution was 0.05 wt % and the final volume of the solution was 50 ml. The mixing was continued for the next 4 min. The final solution had deep rose color. The formation of gold nanoparticles was confirmed by analyzing the UV-vis scan, showing a peak of absorbance at 548.5 nm, which is characteristic for the presence of gold nanoparticles. The solution was then irradiated at 7 kGy, then the UV scan was taken, and the concentration of chlorhexidine gluconate was measured using HPLC. The presence of chlorhexidine in the irradiated sample was not detected, meaning its complete degradation during the standard irradiation procedure which is normally used for sterilization.

### Example 9—Preparation of CHD-Coated Gold Particles by Irradiation Method

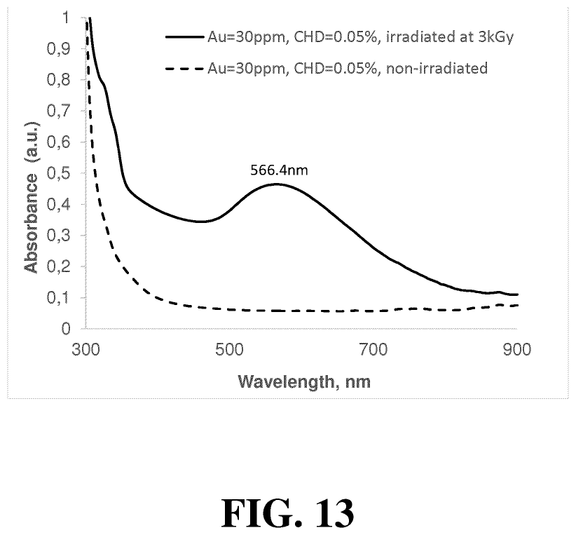

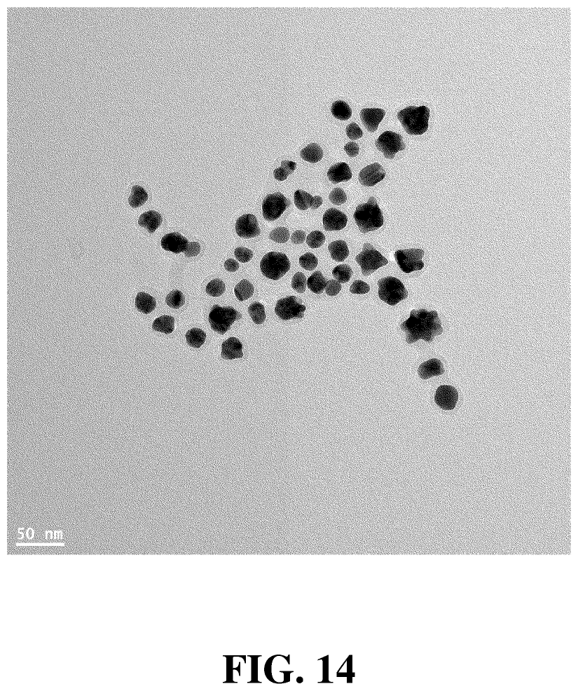

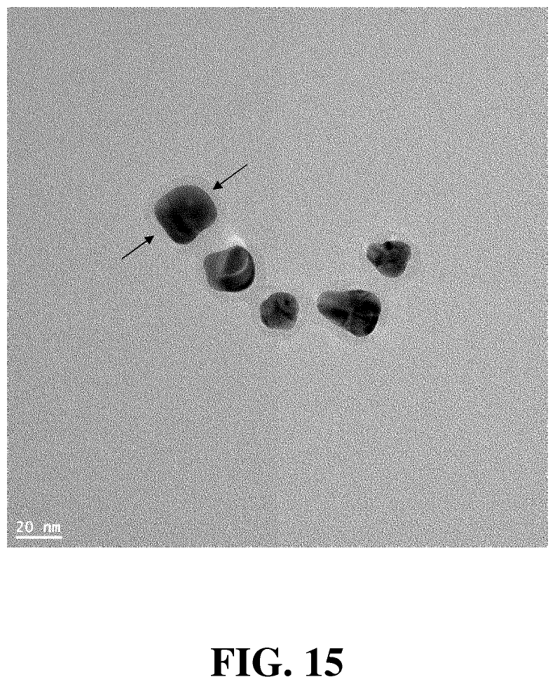

The aqueous solution containing chloroauric acid (from 1 wt % solution of HAuCl₄*3H₂O), isopropanol and chlorhexidine gluconate (from 20 wt % solution in water) was prepared so that the concentration of gold in the resulting solution was 30 ppm, the concentration of isopropanol was 4 wt % and the concentration of chlorhexidine gluconate was 0.05 wt %. The solution was transparent and colorless. The colorless sample was irradiated by gamma-rays at 3 kGy and the resulting solution had transparent dark blue color. Chlorhexidine gluconate was detected at 0.0258 wt %, meaning the preservation of 51.6%. The UV-vis spectra of both samples are compared in FIG. 13. The non-irradiated sample does not show any peaks meaning that the gold nanoparticles were not created. The irradiated sample has a characteristic peak at 566.4 nm, which is representative for the presence of gold nanoparticles. The image of the irradiated solution made by FEI Tecnai G² F20 200 kV Cryo-STEM Transmission Electron Microscope is presented in FIG. 14, where the nanoparticles of quasi-spherical and star-like shapes can be observed; all of them having the size less than 50 nm. It is noticeable that the nanoparticles have a conjugated layer on their surface and its thickness can be estimated as being approximatively 5 nm, as it is shown in FIG. 15, which provides higher magnification of the same nanoparticles as shown in FIG. 14.

While the present technology has been described in connection with specific embodiments thereof, it will be understood that it is capable of further modifications and this application is intended to cover any variations, uses, or adaptations of the invention following, in general, the principles of the present technology and including such departures from the present disclosure as come within known or customary practice within the art to which the present technology pertains and as may be applied to the essential features hereinbefore set forth, and as follows in the scope of the appended claims.

### INCORPORATION BY REFERENCE

All references cited in this specification, and their references, are incorporated by reference herein in their entirety where appropriate for teachings of additional or alternative details, features, and/or technical background.

### EQUIVALENTS

While the disclosure has been particularly shown and described with reference to particular embodiments, it will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following embodiments.

1. A chlorhexidine system comprising:
i) metallic particles, the metallic particles having a core and a surface, and
ii) chlorhexidine or a salt thereof;
wherein the chlorhexidine or the salt thereof is conjugated to the surface of the metallic particles.

2. The chlorhexidine system according to claim 1, wherein the metallic particles comprise a transition metal.

3. The chlorhexidine system according to claim 1, wherein the metallic particles comprise silver.

4. The chlorhexidine system according to claim 1, wherein the metallic particles comprise gold.

5. The chlorhexidine system according to claim 1, wherein the metallic particles comprise silver and gold.

6. The chlorhexidine system according to claim 1, wherein the chlorhexidine system is formed by irradiation.

7. The chlorhexidine system according to claim 6, wherein the irradiation is a gamma irradiation.

8. The chlorhexidine system according to claim 1, wherein the metallic particles are nanoparticles.

9. The chlorhexidine system according to claim 1, wherein the metallic particles have an average size ranging from between about 1 nm and about 1000 nm.

10. The chlorhexidine system according to claim 1, wherein the metallic particles have an average size ranging from between about 1 nm and about 100 nm.

11. A composition comprising:
a) the chlorhexidine system according to claim 1; and
b) at least one additional component.

12. The composition according to claim 11, wherein the at least on additional component is an alcohol.

13. The composition according to claim 11, wherein the at least one additional component is benzalkonium chloride.

14.-16. (canceled)

17. A method for obtaining the chlorhexidine system as defined in claim 1, the method comprising irradiating a mixture of metallic salts and the chlorhexidine or a salt thereof with gamma radiation.

18. The method according to claim 17, wherein the gamma radiation is between about 1 kGy and about 50 kGy.

19. The method according to claim 17, wherein the chlorhexidine system has a rate of preservation of chlorhexidine of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

20.-27. (canceled)
